(19) United States
(12) Patent Application Publication
(10) Pub. No.: US 2012/0041297 A1
(43) Pub. Date: Feb. 16, 2012

(54) REAL-TIME MAGNETIC DIPOLE DETECTION AND TRACKING

(75) Inventor: John E. McGary, Houston, TX (US)

(73) Assignee: Baylor College of Medicine, Houston, TX (US)

(21) Appl. No.: 13/148,125

(22) PCT Filed: Feb. 5, 2010
(86) PCT No.: PCT/US10/23345

§ 371 Date: Sep. 22, 2011

Related U.S. Application Data
(60) Provisional application No. 61/150,512, filed on Feb. 6, 2009.

(51) Int. Cl. A61B 5/05; G01R 33/035; G06F 19/00; G01B 7/14; G01R 33/09

(52) U.S. Cl. 600/409; 324/207.11; 324/207.21; 702/150; 324/207.13

(57) ABSTRACT

A system for detecting a position and/or orientation of a magnetic dipole includes a first detector and a second detector, separate and spaced apart from the first detector. Each of the detectors includes three or more magnetic field sensors to detect a magnetic field generated by the magnetic dipole. The system has applications, for example, in image-guided surgery and/or therapy.

### BACKGROUND

This disclosure relates to real-time magnetic dipole detection and tracking. The ability to detect and track magnetic dipoles can be useful in a number of medical procedures and therapies, such as external beam radiotherapy and computed tomography (CT) imaging. External beam radiotherapy is a procedure in which ionizing radiation (e.g., high energy photons, electrons, or protons) from a linear accelerator is delivered in the form of multiple treatment beams at a tumor or treatment site. Radiation enters the patient body and deposits a particular dose (measured as energy/mass) throughout the treatment volume, where dose is delivered to both the tumor and the surrounding normal tissue. A major goal in radiotherapy is to restrict the dose exposure to the normal tissue and increase the dose within the tumor volume to achieve curative doses.

Geometric uncertainty, however, is a significant problem in external beam radiotherapy and other medical procedures. For example, tumor motion, either due to respiration or skeletal motion, is typically present during treatment delivery and CT imaging. This motion can lead to insufficient radiation doses to the treatment area or excessive radiation doses to normal tissue. To account for this uncertainty, treatment plans are designed to identify a region that the tumor may occupy during treatment so that geometric misses are reduced; however, increased dose is delivered to the surrounding normal tissue and critical structures. Accordingly, it is helpful to detect and track the tumor, or other target, during treatment and treatment planning to reduce normal tissue doses while escalating target doses.

Treatment planning entails obtaining 3D images (e.g., computed tomography (CT) images, magnetic resonance imaging (MRI) images, or ultrasound images) of the patient body and internal organs to prepare the optimal treatment beams and delivery. However, during image acquisition, as well as during treatment, the organs and tumor may move (for example, due to breathing), which can introduce positional uncertainties. 4D computed tomography (4DCT) is a process that gathers patient images over the course of multiple breathing cycles for use in radiation treatment planning for gated radiotherapy delivery. The 4DCT dataset is composed of multiple 3D CT datasets, in which each dataset is intended to represent a specific tumor motion phase. The images provide a time-lapsed 3D (i.e., 4D) image showing tumor motion as well as the motion of nearby organs.

This information obtained from 4DCT allows a practitioner to design a treatment program in which radiation is delivered to a moving target. For instance, a tumor on the lung will move along with each breath as the lung inflates and deflates. Using traditional imaging, an oncologist would know where the tumor is positioned at only one point in the breath. Radiation could then be aimed at the tumor, but the beam would only hit the tumor as intended during the one point of the breath matching the image; at other times, the beam could miss the tumor, or healthy tissue would be irradiated. The information gathered from 4DCT, however, allows for radiation to be delivered to the tumor within a certain interval in the breathing cycle, i.e., gated therapy.

The application of 4DCT with gated therapy is based on the use of external markers placed on the patient's skin to infer the location of the tumor or other targets. These markers are “surrogates” since they do not measure the actual tumor position during the 4DCT scan. Typically, these markers are tracked optically using systems such as, for example, the Varian® Real-time Position Management™ system. Image sets then are obtained by the CT system and binned according to the phase determined from the surrogate markers. For example, the images can be binned according to various respiratory phases (inhale, exhale and in-between) determined by surrogate markers placed on the abdomen. The target location then can be deduced based on the phase of the markers associated with the binned images. However, the surrogate marker phases do not correlate well with the target motion.

To accurately measure the tumor position during 4DCT, an alternative method using magnetic markers can be devised. However, the detection of the magnetic fields emitted by the markers can be limited due to the high noise environment that is generated by the continuous rotation of the gantry and associated components. Likewise, other systems such as TomoTherapy present similar problems due to continuous gantry rotation.

### SUMMARY

Various aspects of the disclosure are set forth in the claims. For example, in one aspect, a system for detecting a position of a magnetic dipole includes a first detector, and a second detector, separate and spaced apart from the first detector, in which each detector includes three or more magnetic field sensors to detect a magnetic field generated by the magnetic dipole.

In another aspect, a target locating and tracking system for use in image guided medical treatments includes a transponder in, on or adjacent to a patient, a first detector, a second detector, separate and spaced apart from the first detector, in which each detector comprises three or more magnetic field sensors to detect a magnetic field generated by the transponder, and a data processing unit configured to receive magnetic field data generated by the magnetic field sensors and to determine the position of the transponder based on the received magnetic field data.

In some implementations, one or more of the following features are present. For example, the data processing unit can be configured to determine the position of the magnetic dipole based on a relative position vector between the first and second detectors.

In some cases, each of the three or more magnetic field sensors is a SQUID magnetometer. Alternatively, or in addition, the magnetic field sensors can be magneto-resistive sensors.

In some circumstances, the first detector is in a first position and the second detector is in a second position substantially opposite the first position. In certain cases, the first detector and the second detector are located at substantially diametrically opposite positions around a treatment position.

In certain implementations, at least three of the magnetic field sensors in each detector are arranged in mutually orthogonal positions. The magnetic field sensors in each detector can be arranged in a cube geometry.

In certain cases, the system includes a third detector, in which the third detector has three or more magnetic field sensors to detect the magnetic field generated by the magnetic dipole.

In some implementations, the target locating and tracking system includes a power transfer circuit external to the patient to generate an oscillating magnetic field for charging the transponder. The power transfer circuit can contain a dipole antenna.

In another aspect, a method of locating a transponder includes supplying power to the transponder to produce an oscillating magnetic field, measuring the oscillating magnetic field in a first detector and in a second separate detector, and calculating a location of the transponder based on measurements of the oscillating magnetic field obtained from the first detector and the second detector.

In some implementations, measuring the oscillating magnetic field in each of the first and second detectors includes detecting the oscillating magnetic field in at least three magnetic field sensors. Detecting the oscillating magnetic field in at least three magnetic field sensors can further include detecting three mutually orthogonal components of the oscillating magnetic field. In some cases, the method of locating a transponder further includes averaging the measurements from the at least three magnetic field sensors in the first detector to provide a first averaged field, and averaging the measurements from the at least three magnetic field sensors in the second detector to provide a second averaged field, in which calculating the location of the transponder is based on the first averaged field and the second averaged field.

In certain embodiments, calculating the location of the transponder is further based on a relative position between the first and second detectors.

In some cases, locating the transponder further includes measuring the oscillating magnetic field in a third detector, in which the third detector is separate and spaced apart from both the first and second detectors.

In some situations, supplying power to the transponder includes passively charging the transponder. Passively charging the transponder can include generating a magnetic field from a power transfer circuit.

The transponder may be placed on, in or adjacent to a patient. In certain implementations, the first detector and second detector can be arranged at substantially opposite positions around a treatment position.

In various implementations, one or more of the following advantages are present. Due to the high sensitivity and flat frequency response, the SQUID sensors and/or magnetoresistive sensors can detect the magnetic dipole signal in magnetically noisy environments. Moreover, the detectors can placed up to about 100 cm away from the marker that contains the source magnetic dipole.

Given the small size of SQUID and magnetoresistive magnetometers, detectors which incorporate such sensors can be positioned within or near a bore of the imaging or treatment device simultaneously with a patient, without interfering with the patient or attenuating incident beam path. Accordingly, the detectors reduce artifacts that lead to degradation in image quality during pre-treatment image analysis as well as reduce excessive patient skin dose that may occur.

Additionally, the detectors can be used to precisely locate and track the position and orientation of magnetic markers within about 3 mm or less. In addition, multiple magnetic dipole sources are not necessary to locate the target. Instead, a target can be tracked using detectors that identify the position and orientation of a single magnetic marker.

Details of one or more embodiments of the invention are set forth in the accompanying drawings and the description below. Other features and advantages will be apparent from the description, the accompanying drawings, and the claims.

### BRIEF DESCRIPTION OF THE DRAWINGS

FIG. 1 illustrates an example of a system used to detect and track the location of a target during a medical procedure.

FIG. 2 shows an example of a marker that can be placed in, on or adjacent to a target to mark the target's location.

FIG. 3 shows an example arrangement of two detectors and a marker configured to emit a magnetic field.

FIG. 4 shows a block diagram illustrating an example process of determining a marker location.

FIG. 5A illustrates a perspective view of a first detector, second detector, a target, a marker and CT device.

FIG. 5B shows a top view of the detector arrangement shown in FIG. 5A.

FIG. 5C shows a side view of the detector arrangement shown in FIG. 5A.

FIG. 6 shows an example of a detector that incorporates a SQUID magnetometer.

FIG. 7 shows an example of a mounting assembly for a detector that incorporates one or more SQUID magnetometers.

FIG. 8 shows an example of a detector that incorporates one or more magnetoresistive sensors.

### DETAILED DESCRIPTION

Magnetic tracking can be used as a method of target detection and tracking in medical procedures. A magnetic marker is placed adjacent to, on, or inside a patient (e.g., within a tumor) and then detected during image acquisition and treatment to enable accurate treatment planning and treatment delivery. Although magnetic tracking can be invasive, the surgical procedure to implant the magnetic marker is minimal and clinically acceptable. Intra-tumoral implants are routinely used in radiotherapy such as: brachytherapy implants that consist of multiple shapes, sizes, delivery techniques, and sources; and radio-opaque fiducials that are made of various metals and shapes are used for patient setup.

The concept of a magnetic-tracking system for real-time tumor localization is based on using implantable transmitters/transponders that generate an oscillating magnetic field, which can be measured by an external detection system. The measured signal then may be used to calculate the transmitter/transponder position and orientation. The implantable transmitter/transponder can be passively energized by an external power source. For example, the transmitter/transponder may be inductively charged by a magnetic field emitted by an external dipole circuit. In response, the transmitter/transponder emits a subsequent magnetic field having the same or different frequency as the field generated by the external dipole circuit. Accordingly, the transmitter/transponder, itself, can function as an oscillating magnetic dipole.

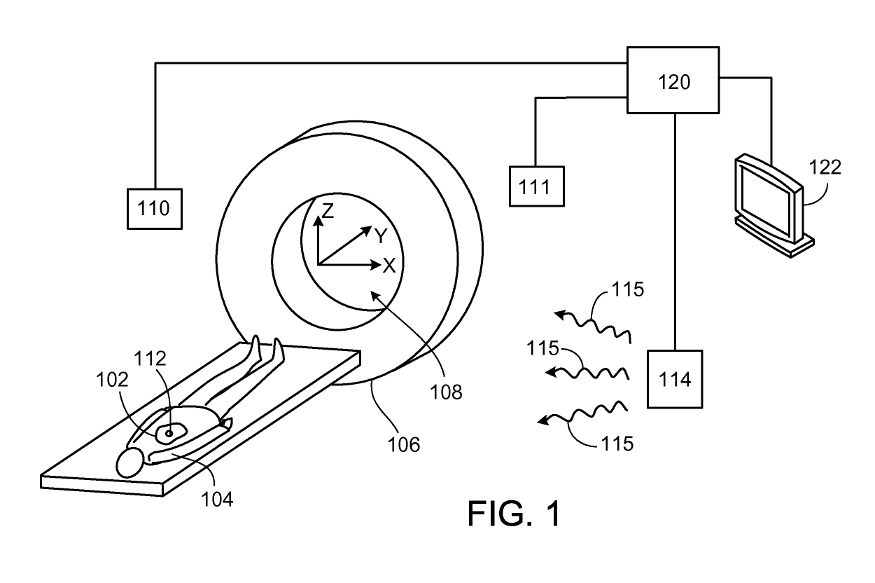

FIG. 1 illustrates an example of a system 100 used to detect and track the location/orientation of a target 102 during treatment planning or during a medical procedure, such as external beam radiotherapy. The system 100 is configured to apply radiation (e.g., X-ray, electron-beam radiation) towards the target 102, such as a tumor, within the body of a patient 104. The system 100 is not limited to external beam radiotherapy or diagnostic imaging, however, and may be utilized in procedures such as image-guided surgery, among others.

The system 100 shown in the example of FIG. 1 allows the target 102 to be located in real-time while radiation from an imaging device 106 is directed at the patient 104. The radiation delivery device 106 is a TomoTherapy assembly in which a radiation source is incorporated within a CT scanning device which rotates around a bore 108. During treatment delivery or treatment planning, the patient 104 is positioned within the bore 108. To prepare for treatment delivery, a treatment plan should be generated for the specific target location. Accordingly, knowledge of the target position during image acquisition and treatment ensures accurate delivery of radiation. Radiation sources other than a TomoTherapy or CT assembly may be used as well.

As radiation from the device 106 is emitted, the target 102 may shift positions due to internal movement, such as organ motion or breathing, or due to external movement of the patient. Using a series of detectors 110, 111, the location and/or orientation of the target 102 can be tracked by measuring a magnetic signal emitted by a marker 112 that is attached or adjacent to the target 102. Although only two detectors are shown in the example system 100 of FIG. 1, additional detectors may be used as well. The marker 112 is a transmitter or transponder that can be energized by an excitation source 114 positioned exterior to the patient's body 104. The excitation source 114 can be a dipole circuit, or other power transfer circuit, arranged to emit an electromagnetic signal 115. The source 114 is operatively coupled to a computer 120 or other device that includes an electronic data processing unit and memory. The computer 120 can be programmed or operated to modify the frequency, signal strength and pattern of the electromagnetic signal emitted by the excitation source 114. The electromagnetic signal emitted by the source 114 may be received by the marker 112 wirelessly or through a wired connection. After the marker 112 is energized, it emits a magnetic field which resonates at a selected frequency and which can be measured by detectors 110, 111. The frequency of the magnetic field emitted by the marker 112 may be equal to or different from the frequency of the excitation source 114.

The detectors 110, 111 can be placed at various locations including: mounted on the floor; mounted from the ceiling; outside the bore 108 on the same side of the device 106; or mounted on opposing sides of the bore 108. In some cases, the detectors 110 also may be placed inside the bore 108 depending on the detector size. For CT systems, the bore 108 includes a conical outer shell. One or more detectors 110 may be placed at an angle inside the bore so as not to interrupt patient movement, gantry operation or the radiation beam path. In TomoTherapy systems, the bore is comparable to wide bore CT systems where the diameter is about 1000 mm. In some applications, the detectors 110 can be mounted to the gantry housing of the gantry rotation device. However, vibrations from the CT device 106 may reduce detector sensitivity should the detectors be mounted on the gantry housing.

The detectors are operatively coupled to the computer 120 such that the computer 120 receives the measurement information obtained from each detector 110, 111. The computer 120 is programmed to include one or more algorithms that, when executed, determine the location and/or orientation of the marker 112, and thus the location and/or orientation of the target 102, based on the measurement information. Once the location and/or orientation information of the marker 112 is determined, the information is stored in memory or output to a peripheral device such as the monitor 122. In some cases, the location and/or orientation information is overlaid with images obtained from the CT device 106 on the monitor 122. As a result, a physician or operator can use the displayed information to direct radiation towards the target 102 with improved accuracy. Alternatively, or in addition, the physician or operator may move the patient 104 and/or target 102 until the target 102 is coincident, within acceptable limits, with the radiation beam.

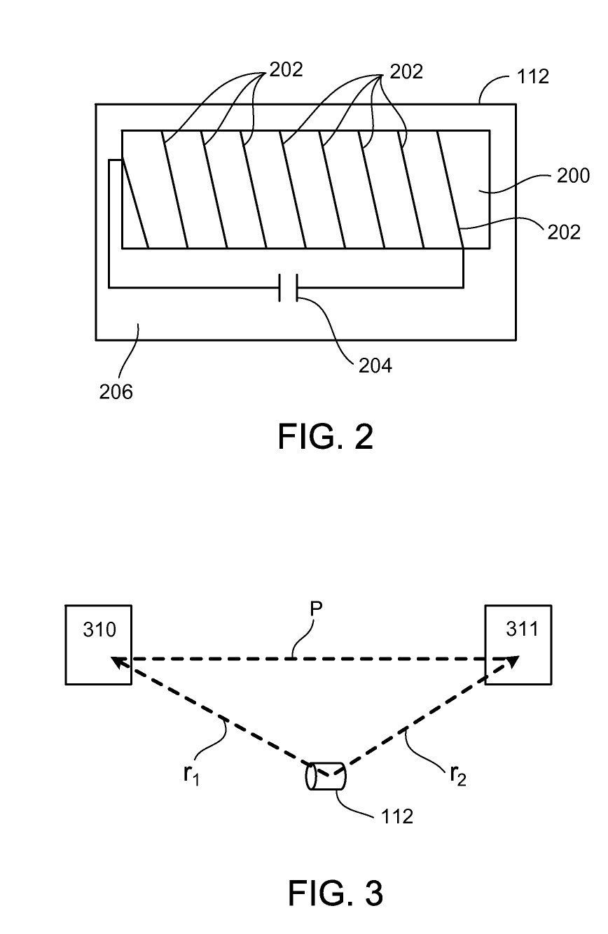

FIG. 2 shows an example of the marker 112 that can be placed in, on or adjacent to the target 102 to mark the target's location. As shown in the example, the marker 112 is a cylindrical resonator, implantable within the patient's body, that includes a ferrite core 200 over which a conductive wiring 202 is wound. The wiring 202 can be connected to a capacitor 204 and/or antenna (not shown). Other passive or active electronic components may be incorporated into the marker 112, as well. When inductively charged by an electromagnetic field emitted from the excitation source 114, the marker 112 resonates at a certain frequency such that a magnetic field emanates from the marker 112. The core 200, winding 202, capacitor 204 and other components of the marker 112 should be encapsulated with a biologically inert coating 206 if the marker 112 is to be implanted within the patient's body or the target 102. The coating 206 serves to prevent the marker 112 from being rejected by the patient's immune system. The coating 206 includes, but is not limited to, materials such as glass. The marker diameter can be about 5 mm (e.g., about 1 mm, 2 mm, 4 mm, 8 mm). The marker length can be about 10 mm (e.g., about 1 mm, 5 mm, 15 mm, 20 mm). In some cases, the excitation source 114 is not necessary, and the marker 112 can be energized by a power source incorporated within the marker 112 itself or elsewhere within the patient body. The design of marker 112 is not limited to the example shown in FIG. 2 and can include other implementations as well. The marker 112 can be designed as a serial transponder device, consisting of circuitry that allows the transponder to store charge and then emit a signal by discharging the current through an oscillator. It is charged passively until a specified charge is accumulated and then discharged. For example, the marker 112 can be charged at a frequency between 10 Hz and 10 kHz. Other charging frequencies may be used as well. The energizing circuit can communicate with the transmitter to stop charging during the discharge process. An advantage of the present system is that multiple magnetic dipole sources or markers are not necessary to locate the target. Instead, a target can be tracked using detectors that identify the position and orientation of only a single magnetic marker.

FIG. 3 shows an example arrangement of two detectors 310, 311 and a marker 112 configured to emit a magnetic field. The first detector 310 is located at a first position, identified by vector r₁, relative to the location of the marker 112. The second detector is located at a second position, identified by vector r₂, also relative to the location of the marker 112. The origin may correspond to the location of the magnetic dipole, which is typically near the center of the device bore. Referring to FIG. 1, the axes are oriented with the Z-axis directed upward, the X-axis directed transversely with respect to the direction of the CT bore, and the Y-axis directed into the bore. As shown in FIG. 3, the relative position vector p denotes the distance between the two detectors, i.e., p=r₂−r₁. Although not necessary, additional detectors may be used as well.

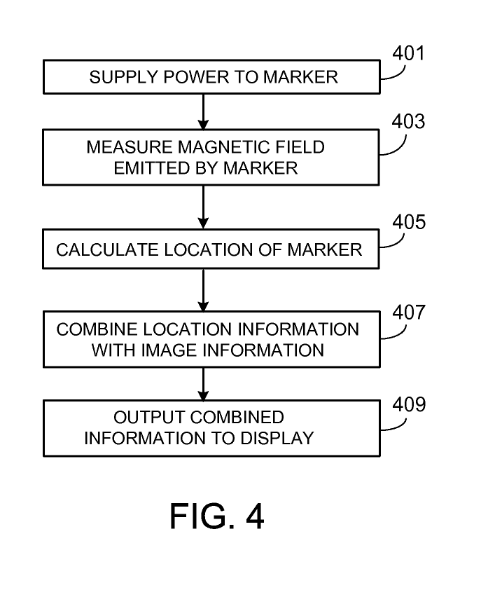

FIG. 4 shows a block diagram illustrating an example process of determining the marker location, and thus the target location, during a CT or 4DCT scan. The process illustrated in FIG. 4 can be performed during other procedures as well, such as external beam radiotherapy conducted in a TomoTherapy system. Power is supplied (401) to the marker 112 using a power transfer circuit such as a dipole circuit that emits a magnetic field with a particular frequency. After being energized by the magnetic field, the marker 112 emits an oscillating magnetic field with the same or different frequency. The oscillating magnetic field emitted by the marker 112 then is measured (403) by the detectors at the same time a CT or 4DCT scan occurs. To determine the position/orientation of the marker 112 in real time, the magnetic field is measured by the detectors at a rate of about 0.1-1 Hz. Other measurement frequencies can be used as well. A location of the marker 112 then is calculated (405) based on the measurements from the detectors. Once the marker location information has been calculated, it can be combined (407) with image information obtained from the CT device 106. The image information can include, for example, images obtained from the CT device 106, information regarding an irradiation beam position or information regarding a reference position of the CT device 106. The combined image information and marker location/orientation information then can be provided (409) to a display, stored in memory or used to calculate a change in an irradiation beam position. For example, a display may show an image of a target obtained from the CT device as well as an icon representing the location of a marker relative to the target. Alternatively, or in addition, the marker location information alone can be provided to a display or stored in memory. In 4DCT systems, the marker location can be overlaid in real-time with target images as they are obtained.

The distance of each detector 110 from the marker 112 can be up to and including about 100 cm (e.g., about 10 cm, 20 cm, 40 cm, 60 cm, 80 cm). The magnetic field measured by the detectors 110 can be modeled using the magnetic dipole equation:

$$B = \frac{{μ}_{0}}{4 π} \frac{3 ( m \cdot n ) n - m}{{r}^{3}} \qquad ( 1 )$$

where B is the magnetic flux density, r is the distance from the dipole source (i.e., marker 112) to the detector 110, n=r/r is the unit vector parallel to the source-detector direction, and m is the magnetic moment. If the relative vector between the location of the detectors 110 is known, two separate detector measurements may be used to solve equation 1. In some cases, measurements of the magnetic field from additional detectors can be used to increase the accuracy of the marker location.

That is, the value of the first detector position r₁ and magnetic moment m can be determined by measuring the magnetic field B₁(r₁) at the location of the first detector, the magnetic field B₂(r₂) at the location of the second detector, and by knowing the relative position vector p between the two detectors 310, 311. For the case of two detectors (e.g., measurements obtained by detectors positioned at two distinct and separate locations) there are 9 equations and 9 unknown variables. The foregoing problem can be solved using Newton's method for root finding by providing an approximation of the initial detector position, r₁.

The value r₁ is obtained by first determining the approximate initial position of the marker relative to the CT device isocenter. In imaging physics and radiation oncology, the isocenter is the intersection point in space through which all central rays of the radiation beams pass for all angles. The approximate marker location can be determined by analyzing CT images which show both the marker and the machine isocenter. Once the relative initial position of the marker is known, the value of r₁ is determined. For example, the value of r₁ can be approximated within about 5 cm or less of the actual detector position. The value of r₁ can be calculated by a processor coupled to the CT device or entered manually by the user.

The marker location/orientation then is determined analytically by solving the system of equations and unknown variables. Accordingly, should the marker/target move, a beam irradiation direction may be adjusted to correct for the change in target position. Alternatively, or in addition, the marker position information can be overlaid with images obtained by the CT device in real time.

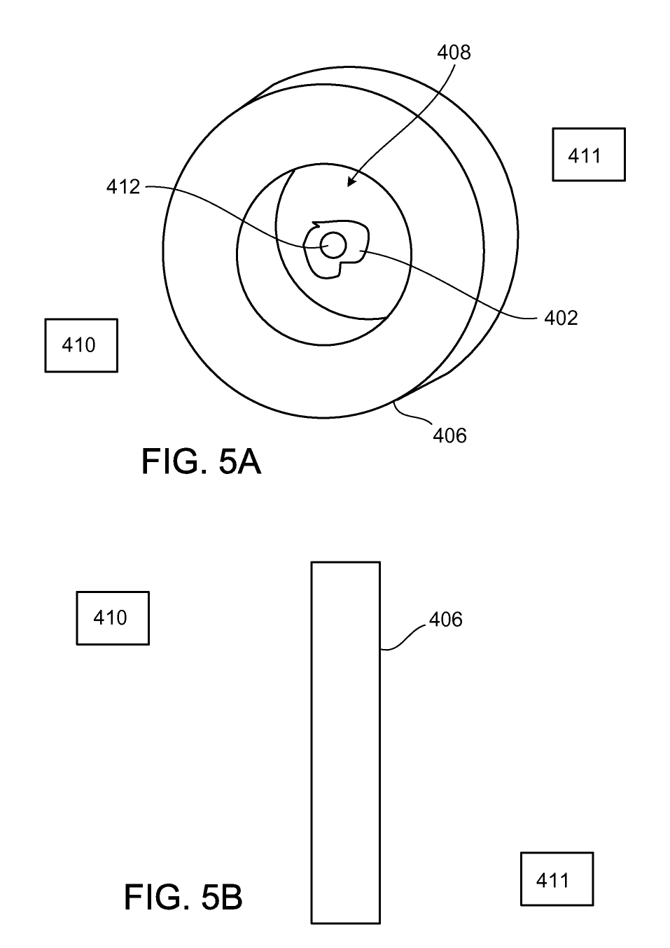

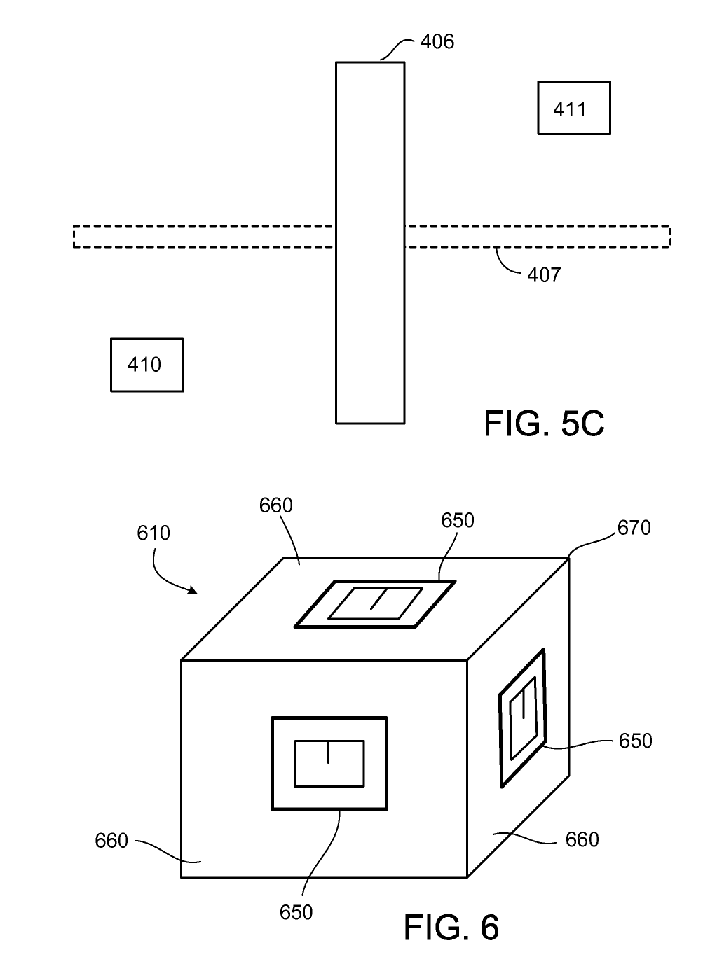

FIG. 5A illustrates a perspective view of a first detector 410, second detector 411, target 402, marker 412 and CT device 406. To facilitate viewing, the patient body is not shown. In some implementations, errors in the calculated marker position can be reduced by placing the two detectors 410, 411 at diametrically opposite positions with respect to the marker 412 (e.g., where r₁ is approximately equal to −r₂). Since an accurate position of the marker 112 is initially unknown, it is preferable to place the detectors at diametrically opposite positions with respect to a fixed treatment location such as the patient's body. Alternatively, or in addition, the detectors can be placed at approximately diametrically opposite positions with respect to the isocenter of the CT device 406.

For example, FIG. 5A shows the first detector 411 located on a first side of the bore 408 whereas the second detector 412 is located on a second opposite side of the bore 408. FIG. 5B shows a top view of the detector arrangement shown in FIG. 5A. FIG. 5C shows a side view of the detector arrangement shown in FIG. 5A. As shown in FIGS. 5A-5C, the detectors 410, 411 are positioned on opposite sides of the bore 408 and diagonally across from each other through the target 402 (not shown in FIGS. 5B, 5C). The outline of a table 407, on which a patient can be supported, is also shown in FIG. 5C. The detectors 410, 411 can be arranged in other configurations as well. For example, in some cases, both the first detector 410 and second detector 411 are located diagonally opposite from each other but on the same side of the bore 408. However, as the detectors 410, 411 are positioned closer together (i.e., the smaller the value of the vector distance p), the error in calculating the marker position may increase. If the vector distance p is not known or cannot be determined, the marker location/orientation still can be calculated using the magnetic field measurements from three or more detectors.

Various types of sensors or magnetometers can be used in the detectors. For example, a detector can include a search coil, a superconducting quantum interference device (SQUID) or a magnetoresistive sensor. Other types of magnetometers may be used as well. With respect to search coils, the coils may be arranged in an array. However, detectors that utilize search coils can limit the detection system in several ways. For example, a typical coil diameter is about 40 mm and a typical array size is about 40 cm×40 cm. Accordingly, the search coil array may be too large to fit within the CT device bore, if needed. Moreover, search coils have a limited sensitivity to magnetic fields and do not exhibit a relatively flat response across the frequency ranges (e.g., 10 Hz-10 kHz) that may be used by the marker. Accordingly, in some cases, the search coils are limited to a distance of about 27 cm from the marker. Moreover, search coils are susceptible to high noise environments, such that they cannot achieve a minimum signal to noise ratio necessary to resolve the magnetic dipole signal emitted by the marker. This can be a particularly significant issue in CT or TomoTherapy systems in which gantry rotation can contribute a noise level of approximately 5 μT.

In addition, search coil arrays can interfere with a CT X-ray beam causing image artifacts and degrading image quality. In certain circumstances, the search coil (when placed in the beam path) may attenuate the irradiation beam, leading to excessive skin dose. Accordingly, a detector that includes a search coil array may not be compatible with all external beam modalities, treatment systems or diagnostic imaging systems.

In contrast, SQUID magnetometers have a high magnetic field resolution and an approximately flat frequency response. In some cases, a SQUID magnetometer may be able to detect magnetic field strengths on the order of tens of ff. Furthermore, SQUID magnetometers have high dynamic range and large slew rates which enables them to detect magnetic signals in high noise environments. Accordingly, SQUID magnetometers can be used in detectors located at distances up to and including about 100 cm.

FIG. 6 shows an example of a detector 610 that incorporates a SQUID magnetometer. The detector 610 is configured in a cube geometry in which a SQUID sensor 650 is mounted on each face 660 of a cube 670. The cube faces can be formed using silicon wafers, printed circuit boards, or other non-magnetic material. The width of each face 660 on the cube can be about 8 mm (e.g., about 2 mm, 4 mm, 6 mm). The magnetic field at the center of the cube can be calculated by averaging the magnetic field measured on opposing cube faces. The marker location/orientation then can be calculated using equation 1.

In some cases, the magnetic field components normal to each face can be used to model the sensor measurement. The underlying system of equations to solve can be described by

$${B}_{k} = \frac{{μ}_{0}}{4 π} \frac{3 ( m \cdot {n}_{k} ) {n}_{k} - m}{{r}_{k}^{3} 30} \qquad ( 2 )$$

where k denotes the cube face identity and Bₖ is the magnetic field vector at the center of the k face. In addition, the position vector for each face, rₖ, is defined as rₖ=r+cₖ, where r is the position vector from the dipole source to the center of the cube and cₖ is the position vector from the center of the cube to the center of face k. For a cube detector having a single SQUID sensor on each face, the problem is well posed with 6 equations that describe the normal field component at each face and 6 unknowns, mᵢ and rᵢ, that represent the magnetic dipole and position vector components. Accordingly, with two cube detectors, the magnetic field can be measured using twelve sensors, i.e., six sensors for each cube detector.

However, it is not necessary that each face of the cube detector include a sensor or even that each detector be formed in the shape of a cube. For example, in the case of two separate detectors, each detector should have a minimum of three sensors positioned orthogonally to one another. Thus, the marker location/orientation can still be calculated by averaging the magnetic field in each detector or by solving the system of equations at each sensor face. In some implementations, the average field technique may be computationally faster when performing real-time calculations.

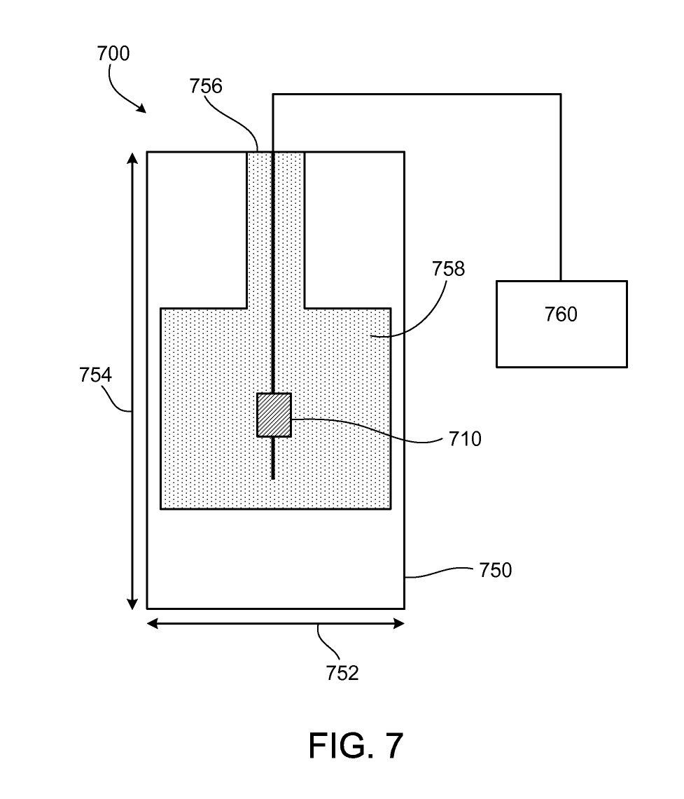

FIG. 7 shows an example of a mounting assembly 700 for a detector 710 that incorporates one or more SQUID magnetometers. As shown in the example, the detector 710 containing SQUID magnetometer(s) can be placed in a cryogenic dewar 750. The dewar 750 can have a cylindrical shape in which the cylinder diameter 752 is about 5 cm (e.g., about 2 cm, 4 cm, 6 cm, 8 cm) and the cylinder length 754 is about 12 cm (e.g., about 10 cm, 14 cm, 16 cm, 18 cm). The dewar 750 also includes an opening 756 to receive the SQUID sensor 740. The dewar 750 may include a cooling liquid 758 such as liquid nitrogen or liquid helium for cooling the SQUID sensors. The detector 710 is electronically coupled to control and noise-cancellation electronics 760 which may be coupled to or contained within a control computer (not shown).

Instead of SQUID sensors, magnetoresistive sensors also can be used in the detectors. Magnetoresistive sensors exhibit a change in material resistivity due to the presence of a magnetic field. Anisotropic magnetoresistive sensors may provide high sensitivity to weak magnetic fields and do not consume a significant amount of energy. When used with feedback electronics, such as flux-lock feedback electronics, magnetoresistive sensors can detect oscillating magnetic fields having frequencies up to about 100 MHz and strengths on the order of about 50 pT or greater. Due to the weaker magnetic field sensitivity than SQUID sensors, magnetoresistive sensors may be located closer to the target/magnetic marker. However, given that magnetoresistive sensors can be incorporated within chip packages (e.g., small outline integrated chip (SOIC), single in-line package (SIP), dual in-line package (DIP)), these sensors can be mounted without interfering with patient movement or the beam path.

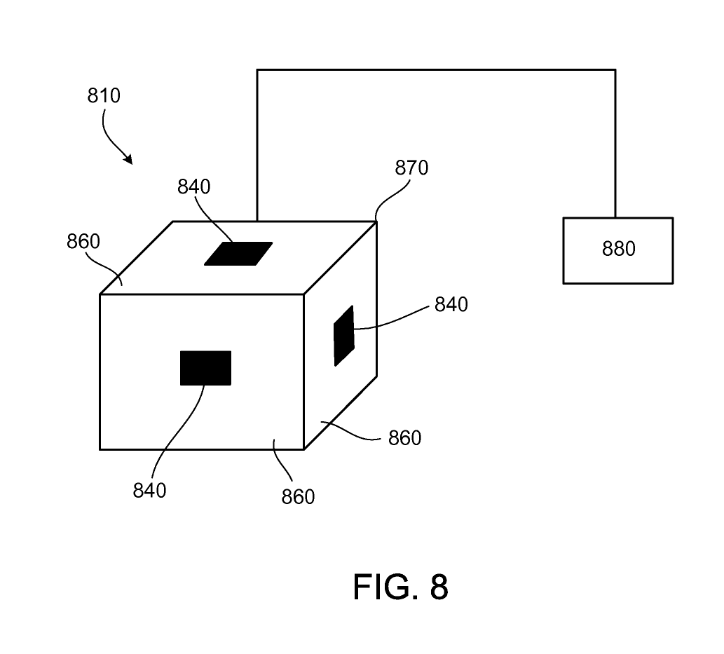

FIG. 8 shows an example of a detector 810 that incorporates magnetoresistive sensors 840. Similar to SQUID sensors, the magnetoresistive sensors can be arranged in a cube geometry or in a pattern in which each sensor is on a plane orthogonal to the planes of the other sensors. For example, the detector 810 is configured in a cube geometry in which the magnetoresistive sensor 840 is mounted on each face 860 of a cube 870. The cube can be fabricated by attaching the sensors to each other. Alternatively, the cube faces may be formed from non-magnetic material such as silicon wafers and non-magnetic circuit boards. Other materials can be used for the cube faces as well. The width of each face 860 on the cube can be about 8 mm (e.g., about 2 mm, 4 mm, 6 mm). The magnetic field at the center of the cube can be estimated by averaging the magnetic field measured on opposing cube faces. In addition, the detector 810 also can be coupled to control and noise-cancellation electronics 880 similar to the electronics used with SQUID sensors. Magnetoresistive sensors do not require, however, the use of cryogenic liquids during operation. Accordingly, in some cases, the magnetoresistive sensors may be smaller than detectors that use SQUID sensors. In addition, the magnetoresistive sensors can be incorporated into a table on which the patient lies during the medical procedure.

Embodiments of the subject matter and the functional operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer program products, i.e., one or more modules of computer program instructions encoded on a computer readable medium for execution by, or to control the operation of, data processing apparatus. The computer readable medium can be a machine-readable storage device, a machine-readable storage substrate, a memory device, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, a runtime environment or a combination of one or more of them.

A computer program (also known as a program, software, software application, script, or code) can be written in any form of programming language, including compiled or interpreted languages, and it can be deployed in any form, including as a stand alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code).

A computer program can be deployed to be executed on one computer or on multiple computers that are located at one site or distributed across multiple sites and interconnected by a communication network.

The processes described in this specification can be performed by one or more programmable processors executing one or more computer programs to perform functions by operating on input data and generating output. Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices.

Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Other implementations are within the scope of the claims.

What is claimed is:

1. A system for detecting a position of a magnetic dipole comprising:
a first detector; and
a second detector, separate and spaced apart from the first detector,
wherein each detector comprises three or more magnetic field sensors to detect a magnetic field generated by the magnetic dipole.

2. The system of claim 1 further comprising a data processing unit configured to receive sensor response signals generated by the magnetic field sensors and to determine the position of the magnetic dipole based on the received sensor response signals.

3. The system of claim 2 wherein the data processing unit is further configured to determine the position of the magnetic dipole based on a relative position vector between the first and second detectors.

4. The system of claim 1 wherein at least one of the three or more magnetic field sensors is a SQUID magnetometer.

5. The system of claim 1 wherein at least one of the magnetic field sensors is a magneto-resistive sensor.

6. The system of claim 1 wherein the first detector is in a first position and the second detector is in a second position and the second position is substantially opposite the first position relative to a treatment location.

7. The system of claim 1 wherein at least three of the magnetic field sensors in each detector are arranged in mutually orthogonal positions.

8. The system of claim 1 wherein the magnetic field sensors in each detector are arranged in a cube geometry.

9. The system of claim 1 further comprising a third detector, wherein the third detector comprises three or more magnetic field sensors to detect the magnetic field generated by the magnetic dipole.

10. A target locating and tracking system for use in image guided medical treatments comprising:
a transponder in or adjacent to a patient;
a first detector;
a second detector, separate and spaced apart from the first detector,
wherein each detector comprises three or more magnetic field sensors to detect a magnetic field generated by the transponder; and
a data processing unit configured to receive magnetic field data generated by the magnetic field sensors and to determine the position and orientation of the transponder based on the received magnetic field data.

11. The target locating and tracking system of claim 10 wherein the data processing unit is further configured to determine the position of the magnetic dipole based on a relative position vector between the first and second detectors.

12. The target locating and tracking system of claim 10 wherein at least one of the magnetic field sensors is a SQUID magnetometer.

13. The target locating and tracking system of claim 10 wherein at least one of the magnetic field sensors is a magneto-resistive sensor.

14. The target locating and tracking system of claim 10 wherein the first detector and the second detector are located at substantially diametrically opposite positions about the patient.

15. The target locating and tracking system of claim 10 wherein at least three of the magnetic field sensors in each detector are arranged in mutually orthogonal positions.

16. The target locating and tracking system of claim 10 wherein the magnetic field sensors in each detector are arranged in a cube geometry.

17. The target locating and tracking system of claim 10 further comprising a third detector, wherein the third detector comprises three or more magnetic field sensors to detect the magnetic field generated by the transponder.

18. The target locating and tracking system of claim 10 further comprising a power transfer circuit external to the patient to generate an oscillating magnetic field for charging the transponder.

19. The target locating and tracking system of claim 18 wherein the power transfer circuit comprises a dipole antenna.

20. A method of locating a transponder comprising:
supplying power to the transponder to produce an oscillating magnetic field;
measuring the oscillating magnetic field in a first detector and in a second separate detector that is spaced apart from the first detector; and
calculating a location and orientation of the transponder based on measurements of the oscillating magnetic field obtained from the first detector and the second detector.

21. The method of claim 20 wherein measuring the oscillating magnetic field comprises detecting the oscillating magnetic field in at least three magnetic field sensors in each of the first and second detectors.

22. The method of claim 21 wherein detecting the oscillating magnetic field in the magnetic field sensors comprises detecting three mutually orthogonal components of the oscillating magnetic field.

23. The method of claim 21 further comprising:
averaging the measurements from the at least three magnetic field sensors in the first detector to obtain a first averaged signal; and
averaging the measurements from the at least three magnetic field sensors in the second detector to obtain a second averaged signal,
wherein calculating the location of the transponder is based on the first averaged signal and the second averaged signal.

24. The method of claim 20 wherein calculating the location of the transponder is further based on a relative position between the first and second detectors.

25. The method of claim 20 further comprising measuring the oscillating magnetic field in a third detector, wherein the third detector is separate and spaced apart from both the first and second detectors.

26. The method of claim 20 wherein supplying power to the transponder comprises passively charging the transponder.

27. The method of claim 26 wherein passively charging the transponder comprises generating a magnetic field from a power transfer circuit.

28. The method of claim 20 further comprising placing the transponder on or adjacent to a patient.

29. The method of claim 20 further comprising arranging the first detector and second detector at substantially opposite positions about a treatment position.

30. The method of claim 20 further comprising wherein measuring the oscillating magnetic field

31. A target locating and tracking system for simultaneously imaging a target and detecting a position of a transponder in or adjacent to the target comprising:
a radiation delivery system configured to obtain images of the target;
a first detector;
a second detector, separate and spaced apart from the first detector,
wherein each detector comprises three or more magnetic field sensors to detect a magnetic field generated by the transponder; and
a data processing unit configured to receive magnetic field data generated by the magnetic field sensors and to determine the position and orientation of the transponder based on the received magnetic field data relative to the images obtained by the radiation delivery system.

32. The target locating and tracking system according to claim 31 wherein the radiation delivery system is a computed tomography system.

33. The target locating and tracking system according to claim 32 wherein the computed tomography system is a 4D computed tomography system.

34. The target locating and tracking system according to claim 32 wherein the computed tomography system is a TomoTherapy system.

35. The target locating and tracking system according to claim 31 wherein the radiation delivery system comprises an image-guided radiation therapy system.

36. The target locating and tracking system according to claim 31 wherein at least one of the magnetic field sensors is a SQUID magnetometer.

37. The target locating and tracking system according to claim 31 wherein at least one of the magnetic field sensors is a magnetoresistive sensor.
